(19) United States
(12) Patent Application Publication
(10) Pub. No.: US 2026/0174328 A1
(43) Pub. Date: Jun. 25, 2026

(54) OPTICAL IMAGING SYSTEM AND OPTICAL IMAGING ANLAYSIS METHOD

(71) Applicant: ELECTRONICS AND TELECOMMUNICATIONS RESEARCH INSTITUTE, Daejeon (KR)

(72) Inventor: Sang Kyun LEE, Daejeon (KR)

(21) Appl. No.: 19/405,849

(22) Filed: Dec. 2, 2025

(30) Foreign Application Priority Data
Dec. 19, 2024 (KR) 10-2024-0191226
Sep. 30, 2025 (KR) 10-2025-0143105

(51) Int. Cl. A61B 3/14

(52) U.S. Cl. CPC A61B 3/14; A61B 2576/026

(57) ABSTRACT

An optical imaging system according to an embodiment disclosed in the present document may include: a laser module configured to radiate an ultrashort pulse laser beam; a microscope module configured to transmit the radiated ultrashort pulse laser beam to an eye of a subject and detect reflected light from the eye or (auto)fluorescence in the eye using an image sensor; a control module configured to generate a tau-related image in which a modification of tau proteins is detectable from the reflected light detected through the image sensor; and an output device configured to display the tau-related image.

### CROSS-REFERENCE TO RELATED APPLICATIONS

This application claims priority to and the benefit of Korean Patent Application No. 10-2024-0191226, filed on Dec. 19, 2024and Korean Patent Application No. 10-2025-0143105, filed on Sep. 30, 2025, the disclosure of which is incorporated herein by reference in its entirety.

### BACKGROUND

### 1. Field of the Invention

Various embodiments disclosed herein relate to optical imaging-based disease diagnosis technology.

### 2. Discussion of Related Art

Alzheimer's disease, one of the representative neurodegenerative diseases, is known to be caused primarily by the abnormal activities of two types of proteins. Specifically, one of the abnormal activities involves the aggregation of Aβ proteins to form insoluble amyloid-β plaques, and the other involves the aggregation of tau proteins to form tau tangles (Neurofibrillary Tangles; NFTs). Accordingly, histological and pathological characteristics of the brain of a patient with Alzheimer's disease are different from those of a normal individual, and such differences are utilized in the diagnosis of Alzheimer's disease.

For example, Alzheimer's disease can be diagnosed at an early stage through methods such as cerebrospinal fluid (CSF) tests (Aβ42/Aβ40, total tau, and p-tau181) and amyloid positron emission tomography (PET). However, PET examination requires the use of a highly expensive equipment, which can impose a financial burden on both the examiner and the examinee.

In recent years, early diagnostic methods for Alzheimer's disease using blood samples have been actively studied, and diagnostic kits have been commercialized and are being used in hospitals as a means for disease screening.

Another example has disclosed a technique in which curcumin is administered into a subject's vein to stain Aβ peptides with a curcumin fluorescent marker, and Alzheimer's disease is diagnosed by detecting the curcumin-bound Aβ peptides from the retinal imaging methods.

### SUMMARY OF THE INVENTION

The disclosure is directed to providing an optical imaging system and an optical imaging analysis method that are capable of generating optical images related to Alzheimer's disease classification based on an ultrashort pulse laser beam and two-photon excitation microscope structure.

According to an aspect of the disclosure, there is provided an optical imaging system according to an embodiment disclosed in the present document, which includes: a laser module configured to radiate an ultrashort pulse laser beam; a microscope module configured to transmit the radiated ultrashort pulse laser beam to an eye of a subject and detect reflected light from the eye or (auto)fluorescence in the eye using an image sensor, a control module configured to generate a tau-related image in which modification of tau proteins is detectable based on the reflected light or (auto)fluorescence detected through the image sensor; and an output device configured to display the tau-related image.

According to another aspect of the disclosure, there is provided an optical imaging analysis method that is performed by an optical imaging system including a laser module, a microscope module, and a control module and includes: radiating an ultrashort pulse laser beam through the laser module and then transmitting the radiated ultrashort pulse laser beam to an eye of a subject through the microscope module; detecting reflected light from the eye or (auto)fluorescence in the eye generated by the transmitted ultrashort pulse laser beam using an image sensor; generating a tau-related image in which modification of tau proteins is detectable based on the (auto)fluorescence or reflected light detected through the image sensor; and determining whether Alzheimer's disease is present by analyzing the generated tau-related image.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other objects, features and advantages of the disclosure will become more apparent to those of ordinary skill in the art by describing exemplary embodiments thereof in detail with reference to the accompanying drawings, in which:

FIG. 1 is a schematic diagram illustrating a configuration of an optical imaging system according to an embodiment of the disclosure.

FIG. 2 is a diagram illustrating a detailed configuration of an optical imaging system according to an embodiment of the disclosure;

FIG. 3 shows brain positron emission tomography (PET) images of a live wild-type (WT) mouse group (normal group) and an AD-Tg mouse group according to age;

FIG. 4 shows a brain image of a mouse captured using an optical imaging system according to an embodiment of the disclosure;

FIG. 5 shows images of Aβ plaques in the brains of 3-month-old AD-Tg mice and WT mice stained with Thioflavin-S, and an images of p-tau404 in the brain stained with a double-positive anti-human PHF-1 antibody;

FIG. 6 shows images of Aβ plaques in the brains of a 9-month-old AD-Tg (5×FAD) mice and a 9-month-old WT mice stained with Thioflavin-S, and images of p-tau404 in mouse brain stained with a double-positive anti-human PHF-1 antibody;

FIG. 7 shows an image in which an Aβ plaque image of the brain of a 16-month-old AD-Tg (5×FAD) mouse stained with Thioflavin-S and a p-tau404 image of the brain stained with a double-positive anti-human PHF-1 antibody overlapping the Aβ plaque image;

FIG. 8 shows an Aβ plaque image of the brain of a 16-month-old AD-Tg (5×FAD) mouse stained with Thioflavin-S, and a Tau tangle (NFT) image of the brain stained with a double-positive anti-human Tangle antibody;

FIG. 9 is a diagram for comparing optical images of Aβ and p-tau404 in the brain of an 11-month-old AD-Tg mouse;

FIG. 10 shows an image of a mouse eye captured by the optical imaging system according to an embodiment;

FIG. 11 shows an image of p-tau404 in the retina of a 5-month-old AD-Tg (5×FAD) mouse stained with a double-positive anti-human PHF-1 antibody;

FIG. 12 shows an image of p-tau404 in the retina of a 9-month-old AD-Tg (5×FAD) mouse stained with a double-positive anti-human PHF-1 antibody;

FIG. 13 shows p-tau404 images in the retinae of 13-month-old and 17-month-old AD-Tg mice (5×FAD) stained with a double-positive anti-human PHF-1 antibody;

FIG. 14 shows the result of fluorescent spot analysis for a p-tau404 image 1400 in the retina of a 13-month-old AD-Tg (5×FAD) mouse stained with a double-positive anti-human PHF-1 antibody; and

FIG. 15 is a flowchart illustrating an optical imaging analysis method according to an embodiment.

In the description of the drawings, the same or similar reference numerals may be used to refer to the same or similar components.

### DETAILED DESCRIPTION OF EXEMPLARY EMBODIMENTS

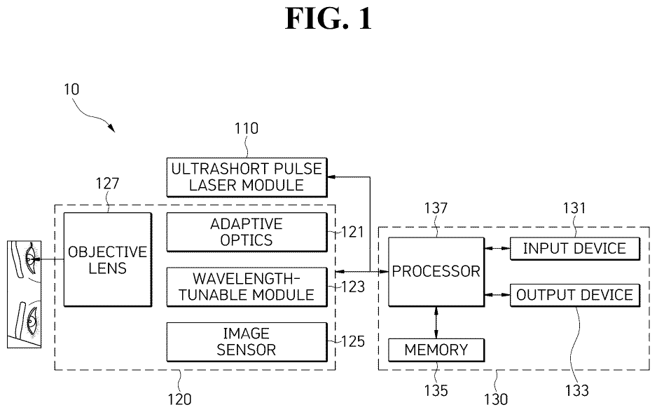

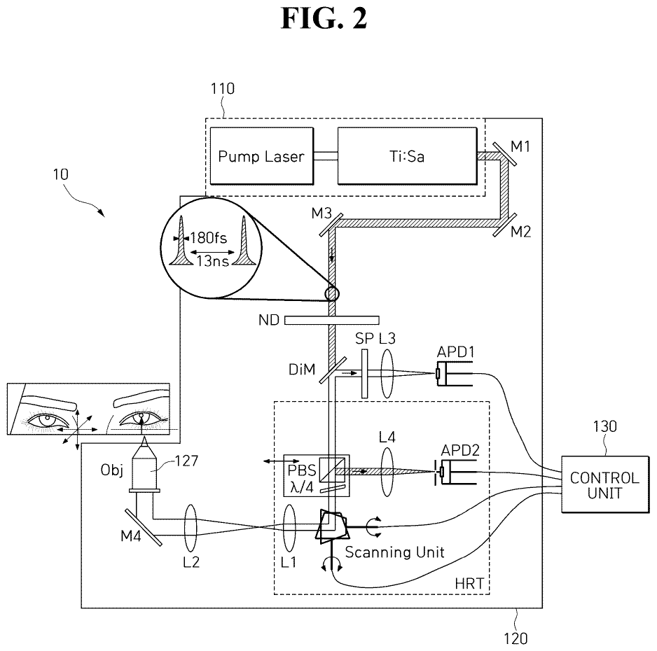

FIG. 1 is a schematic diagram illustrating a configuration of an optical imaging system according to an embodiment of the disclosure, and FIG. 2 is a diagram illustrating a detailed configuration of an optical imaging system according to an embodiment of the disclosure.

Referring to FIG. 1, an optical imaging system 10 according to the embodiment may include an ultrashort pulse laser module 110, a two-photon excitation microscope module 120, and a control module 130. In an embodiment, some components of the optical imaging system 10 may be omitted, or additional components may be further included in the optical imaging system 10. In addition, some of the components of the optical imaging system 10 may be combined into a single entity while still performing the same functions as those of the components before the combination.

As shown in FIG. 2, the ultrashort pulse laser module 110 may include a pump laser using two blue diode lasers and a Ti:sapphire (Ti:Sa) crystal. The Ti:Sa crystal may generate femtosecond pulses with a specified pulse width by amplifying multi-wavelength light through laser oscillation and performing self-mode locking.

An ultrashort pulse laser beam may be light having a central wavelength between 790 nm and 810 nm and multiple wavelengths within a specified wavelength bandwidth. The specified wavelength bandwidth may be less than 11 nm. The pump laser may emit laser beams having a pulse width between 50 fs and 65 fs at a repetition rate between 80 MHz and 110 MHz.

The light radiated through the ultrashort pulse laser module 110 may be incident on the two-photon excitation microscope module 120.

Referring to FIG. 2, the two-photon excitation microscope module 120 may include a wavelength-tunable module 123, an adaptive optics 121, an image sensor 125, and an objective lens 127. The two-photon excitation microscope module 120 may further include additional lenses and mirrors configured to convert (reflect/pass) the path of the ultrashort pulse laser beam.

The adaptive optics 121 may identify a phase shift of the ultrashort pulse laser beam and correct the identified phase shift to focus the ultrashort pulse laser beam incident on the objective lens 127.

The wavelength-tunable module 123 may selectively perform first filtering on the multi-wavelength ultrashort pulse laser beams to extract a laser beam in a first wavelength band in which p-tau404 is most effectively excited under the control of the control module 130 and transmit the first-filtered laser beam to the image sensor 125.

In addition, the wavelength-tunable module 123 may selectively perform second filtering on the multi-wavelength ultrashort pulse laser beams to extract a laser beam in a second wavelength band by which amyloid beta is most effectively excited under the control of the control module 130 and transmit the second-filtered laser beam to the image sensor 125.

When acquiring the laser beam of the first wavelength band through the wavelength-tunable module 123, the image sensor 125 may generate, under the control of the control module 130, an optical image (first optical image) in the first wavelength band in which autofluorescence of the p-tau404 protein may be detected. The first wavelength band may be experimentally set to detect the (auto)fluorescence of the p-tau404 protein.

In addition, when acquiring the laser beam of the second wavelength band through the wavelength-tunable module 123, the image sensor 125 may generate, under the control of the control module 130, an optical image (second optical image) in the second wavelength band in which the (auto)fluorescence of amyloid beta may be detected. The second wavelength band may be experimentally set to detect the (auto)fluorescence of amyloid beta.

The control module 130 may be a computing device (e.g., a PC or laptop computer) configured to acquire the generated images (the first and second optical images) of the two-photon excitation microscope module 120. The control module 130 may include an input device 131, an output device 133, a memory 135, and a processor 137.

The input device 131 may receive an input from a user using the optical imaging system 10. For example, the input device 131 may include at least one input sensing circuit among a button and a touchscreen. The user input may be an operation to start optical image acquisition.

The output device 133 may visually output at least one type of data among symbols, numbers, or characters under the control of the control module 130. For example, the output device 133 may include at least one output device among a liquid crystal display, an OLED display, and a touchscreen display. The output device 133 may output the first and second optical images.

The memory 135 may include various types of volatile and non-volatile memories. For example, the memory 135 may include a read-only memory (ROM) and a random access memory (RAM). In an embodiment, the memory 135 may be located inside or outside the processor 137, and the memory 135 may be connected to the processor 137 through various means already known in the art. The memory 135 may store various types of data used by at least one component (e.g., the processor 137) of the optical imaging system 10. The data may include, for example, input or output data for software and instructions related thereto. For example, the memory 135 may store at least one instruction and data for the optical imaging function. The memory 135 may have Imaris software installed thereon, which is capable of editing/compositing optical images.

The processor 137 may control at least one other component (e.g., a hardware or software component) of the optical imaging system 10 and may perform various data processing or computational operations. For example, the processor 137 may include at least one of a central processing unit (CPU), a graphics processing unit (GPU), a microprocessor, an application processor, an application-specific integrated circuit (ASIC), or a field programmable gate array (FPGA), and may have multiple cores. Although the operations of the control module 130 are performed under the control of the processor 137 according to the operation of an operator, the following description will be made with reference to the control module 130 as the subject.

According to an embodiment, the control module 130 may acquire a first optical image including the intensity of the laser beam in the first wavelength band and a second optical image including the intensity of the laser beam in the second wavelength band from the image sensor 125.

The control module 130 may convert a color of the first optical image into a first color (e.g., red) matched to the first wavelength band to generate a p-tau404 image. The control module 130 may store the p-tau404 image in the memory 135 or output the same through the display (output device 133).

The control module 130 may convert a color of the generated optical image of the second wavelength band into a second color (e.g., green) matched to the second wavelength band to generate an Aβ plaque image. The control module 130 may store the Aβ plaque image in the memory 135 or output the same through the display (output device 133).

In this regard, when the p-tau404 protein and Aβ plaques are exposed to the ultrashort pulse laser beam, they may spontaneously emit fluorescence. By utilizing this characteristic, the optical imaging system 10 according to the embodiment may selectively filter a laser beam in the first and second wavelength bands in which the fluorescence indication of the p-tau protein becomes clear without administering an invasive agent to the subject to transmit the corresponding laser beams to the image sensor 125, thereby generating the p-tau404 image in which the p-tau404 protein is fluorescently visualized and an Aβ plaque image in which the Aβ plaques are fluorescently visualized. However, in various embodiments, at the initial stage of a test using the optical imaging system 10, the invasive agent may be administered to the subject to set (or determine) the first and second wavelength bands by which the autofluorescence of p-tau404 and Aβ plaques may be detected, depending on the characteristics of the environment where the optical imaging system 10 is installed.

The control module 130 may determine whether the subject has Alzheimer's disease by analyzing the size, location, and distribution of fluorescent spots in the retina of the p-tau404 image. Similarly, the control module 130 may determine whether the subject has Alzheimer's disease by analyzing the size, location, and distribution of Aβ plaque fluorescent spots in the retina of the Aβ plaque image.

Meanwhile, the control module 130 may train an AI model with the size, location, and distribution of the fluorescent spots in each retina of the p-tau404 image and the Aβ plaque image. In this case, the control module 130 may determine whether the subject has Alzheimer's disease based on the retinal image of the subject using the trained AI model.

According to the above-described embodiment, the two-photon excitation microscope module 120 further includes optical units M1 to M4 and L1 to L4, a neutral density filter ND and a DIM filter DIM. The optical units M1 to M4 and L1 to L4 may include mirrors and lenses that adjust the optical paths of the laser beam and reflected light so that the ultrashort pulse laser beam is directed toward the objective lens 127 and the reflected light is directed toward the DIM filter DIM. The ND filter ND may adjust the total amount of the ultrashort pulse laser beam to a level that does not cause damage to the subject's eye. The DIM filter DIM may transmit (or pass) the laser beam emitted from the ultrashort pulse laser module 110 and reflect the reflected light from the subject's eye toward the image sensor 125.

In addition, the two-photon excitation microscope module 120 further includes a photomultiplier tube and may detect the total intensity of reflected light from the subject using the photomultiplier tube. In this case, the control module 130 may measure the intensity of a shape signal corresponding to a fluorescent spot based on the total intensity of reflected light detected through the photomultiplier tube.

According to various embodiments, the optical imaging system 10 may further analyze the size, location, and distribution of microglia by fluorescently labeling the Iba1/AIF-1 monoclonal antibody. For example, the optical imaging system 10 may selectively filter a laser beam in a third wavelength band in which the Iba1/AIF-1 monoclonal antibody is excited using the wavelength-tunable module 123 and may generate a microglial cell image in which the Iba1/AIF-1 monoclonal antibody is fluorescently labeled through the image sensor 125. In this regard, similar to p-tau404, the Iba1/AIF-1 protein is not expressed in WT mice in the microglial cell image stained with IBA 1, but may be expressed in the retinal images of 9-month-old AD-Tg Mice (5×FAD).

According to various embodiments, the optical imaging system 10 may further analyze the size, location, and distribution of Tau tangles by fluorescently labeling a Tau (neurofibrillary tangles marker) monoclonal antibody. For example, the optical imaging system 10 may selectively filter a laser beam in a fourth wavelength band in which the Tau monoclonal antibody is excited using the wavelength-tunable module 123 and generate an image of Tau tangles in which the Tau monoclonal antibody is fluorescently labeled through the image sensor 125. For example, the third and fourth wavelength bands may be set, through experiments, to wavelength bands in which the stained microglial cell and the Tau monoclonal antibody are detectable.

As described above, the optical imaging system 10 according to the embodiment, without staining the Aβ plaques, p-tau, and microglial cell in vivo of the examinee with a biologically invasive method (e.g., by administration of an agent), may generate an optical image that determines whether an examinee (or subject) has Alzheimer's disease by detecting, through the ultrashort pulse laser module 110 and the two-photon excitation microscope module 120, not only Aβ plaques caused by the ultrashort pulse laser beam but also spontaneous fluorescence (autofluorescence) of p-tau and microglial cell.

In this regard, according to research results on Alzheimer's disease, amyloid-beta (Aβ) is known not to affect cognitive function, whereas the tau protein is known to affect cognitive function. In addition, according to the diagnostic criteria for Alzheimer's disease announced by the National Institute on Aging and the Alzheimer's Association (NIA-AA). Alzheimer's disease is diagnosed when a condition in which both amyloid (A) and tau (T) are present. In an embodiment, since both the Aβ plaque and the p-tau can be identified, Alzheimer's disease can be more accurately diagnosed.

In addition, the optical imaging system 10 according to the embodiment may detect p-Tau404, which is expressed in the early stage of Alzheimer's disease, among the p-Tau proteins, and thus supports earlier diagnosis of the Alzheimer's disease.

Furthermore, the optical imaging system 10 according to the embodiment may perform non-invasive examinations and repeated measurements, thereby enabling early diagnosis of Alzheimer's disease, and reducing the examination burden on both the subject and the examiner by simplifying the overall examination process.

Furthermore, the optical imaging system 10 according to the embodiment may be configured at a lower cost than that of an expensive PET examination device, which is implemented in a form for seeing through the skull for brain diagnosis and has risks of radiation exposure as well as potential side effects such as headache or hemorrhage, thereby facilitating commercialization and reducing side effects on the subject.

Hereinafter, the effects of the optical imaging system 10 according to the embodiment will be described based on experimental data.

The characteristics of the AD-Tg (5×FAD) mouse known in the academic field are as follows:
1) Aβ plaques begin to form after 2 months of age;
2) From 3 months of age, the mouse exhibits mild cognitive impairment and neuronal loss;
3) After 5 months of age, the mouse exhibits mild dementia and significant Aβ plaque accumulation, leading to severe memory loss;
4) From 7 months of age, the mouse exhibits severe dementia and cognitive and spatial memory deficits; and
5) From 10 months of age, Aβ levels peak, leading to neuronal cell death and severe neuronal loss.

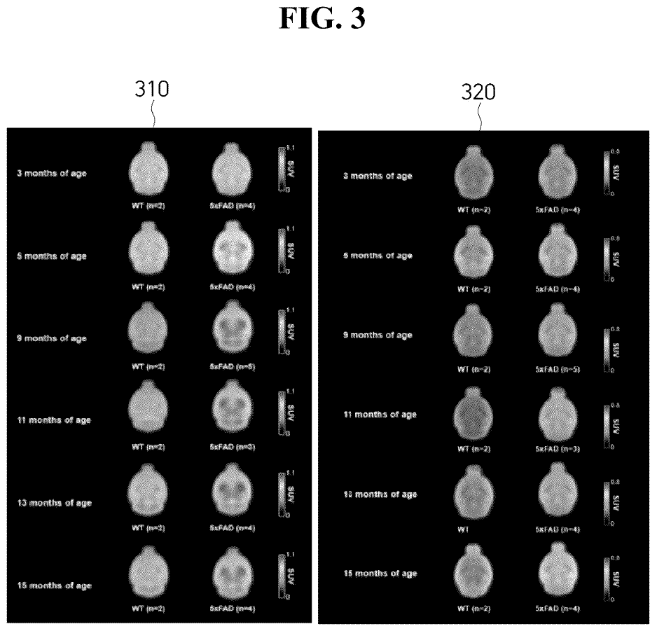

Hereinafter, with reference to FIG. 3, the characteristics of the AD-Tg mouse in conventional positron emission tomography (PET) images will be described. FIG. 3 shows brain PET images of a live wild-type (WT) mouse group (normal group) and an AD-Tg mouse group according to age.

Amyloid-PET images 310 shown on the left side of FIG. 3 was taken 30 to 60 minutes after injection of the 18F-florbetaben agent and are PET images of amyloid in the brains of WT (wild-type) mice (normal group) and AD-Tg mice according to age.

In the amyloid-PET images 310, at 3 months of age, there is almost no difference in Aβ accumulation between the two groups (that is, WT mice and AD-Tg mice), and it can be seen that no significant accumulation changes are observed even in major brain regions such as the cerebral cortex, thalamus, and hippocampus. In other words, it can be seen that Aβ accumulation at 3 months of age remains at an early stage.

However, from 5 months of age, it can be seen that Aβ accumulation in the thalamus of the AD-Tg (5×FAD) mouse group begins to increase compared with the normal group, thereby confirming that the thalamus may be a major region of early amyloid accumulation.

In addition, in the 9-month PET images (at 9 months of age), it can be seen that Aβ accumulation in the thalamus and hippocampus of the AD-Tg (5×FAD) mouse group becomes significantly increased from 9 months of age, so that Aβ accumulation begins to progress substantially at 9 months of age. In summary, it can be seen that, in the AD-Tg (5×FAD) mouse group, Aβ accumulation in specific brain regions begins to occur intensively from 9 months of age.

From 11 months to 15 months of age (at 11, 13, and 15 months of age), the PET images of the AD-Tg (5×FAD) mouse group show a relatively similar pattern of Aβ accumulation compared with the previous stages. In particular, after 13 months of age, the level of Aβ accumulation in the AD-Tg (5×FAD) mouse group appears to reach a saturation state. However, in the normal group, Aβ accumulation tends to slightly increase due to nonspecific changes associated with aging.

A tau-PET image 320 shown on the right side of FIG. 3 was taken 30 to 60 minutes after injection of the 18F-T807 agent and is a PET image of tau in the brains of WT mouse group and AD-Tg mouse group according to age.

In the tau-PET images 320, it can be seen that, at 3 months of age, there is no observable difference in tau accumulation between the normal group (WT) and the AD-Tg (5×FAD) mouse group in the major brain regions including the cerebral cortex, thalamus, and hippocampus. That is, it reflects the pathological characteristics (known characteristics) of the early stage of Alzheimer's disease (AD) in which the accumulation of tau protein is minimal in the initial phase.

However, in the PET images at 5 months of age of the mice, it can be seen that tau protein accumulation in the thalamus of the AD-Tg (5×FAD) mouse begins to increase compared with the normal group. Similar to the amyloid-PET images, the tau-PET images also suggest that the thalamus is an early manifestation region of tau accumulation and may be an important region in the progression of AD pathology.

In the PET images at 9 months of age, since tau protein accumulation in the thalamus and hippocampus becomes more distinct from 9 months of age, it can be seen that tau pathology begins to progress in earnest at this stage. At this time, it can be seen that tau accumulation in the AD-Tg (5×FAD) mice begins to spread to major brain regions.

At 11 months of age, it can be seen that tau accumulation in the cerebral cortex begins to increase noticeably, and that accumulation in the thalamus and hippocampus becomes more significant. That is, this reflects the stage in which the progression of Alzheimer's disease (AD) begins to affect multiple brain regions simultaneously from 11 months of age.

After 13 months (at 13 and 15 months of age), tau accumulation in the cerebral cortex, thalamus, and hippocampus of the AD-Tg (5×FAD) mouse shows a pattern approaching its maximum level. Since such changes are not observed in the normal group, this suggests that tau accumulation is closely related to the progression of AD pathology.

At 15 months of age, it can be seen that tau protein accumulation in the AD-Tg (5×FAD) mice remains at a high level, whereas no significant accumulation pattern is observed in the normal group.

However, in the PET images of FIG. 3, since the scale bar of 0-0.8 of the tau-PET images 320 is slightly smaller than the scale bar of 0-1.1of the amyloid-PET images 310, a difference in PET uptake of 18F-T807 between the normal group and the AD-Tg group may be relatively smaller than that of 18F-florbetaben.

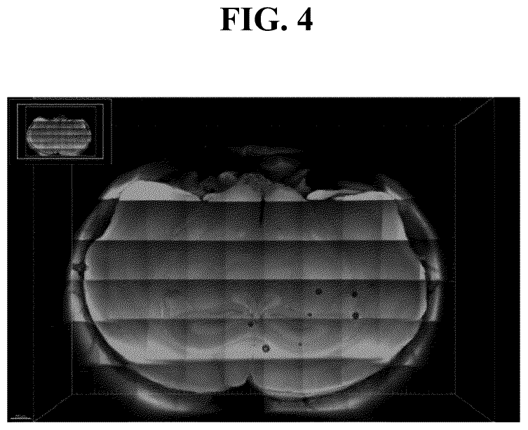

Hereinafter, with reference to FIGS. 4 to 8, the effects of the optical imaging system 10 according to the embodiment will be described based on the optical images of the brains of experimental mice captured through the optical imaging system 10. The optical images of FIGS. 4 to 8 were obtained by capturing the brains of AD-Tg mice and WT mice that were subjected to fluorescence staining and tissue clearing processes. However, since the brain of the mouse is surrounded by the skull and the ultrashort pulse laser light radiated by the optical imaging system 10 cannot transmit the skull, the brain of the mouse was extracted after sacrifice (pre-treatment) and then imaged. In addition, the extracted mouse brain was immersed in a Thioflavin-S solution, subjected to fluorescence staining of Aβ, and a tissue clearing process was subsequently applied using a tissue clearing solution. The brains extracted from WT mice of the same age group were also subjected to the same staining and tissue clearing processes. The double-positive anti-human PHF-1 antibody was also stained after the Thioflavin-S staining and tissue clearing processes. The sample (mouse brain) was prepared with a thickness of 1.5 mm to obtain the entire image of the hippocampal region, and fluorescence images were also acquired as z-stacks of 1.5 mm thickness with 2 μm intervals. In this regard, the optical imaging system 10 may detect the autofluorescence of abnormal tau proteins within the mouse brain through the two-photon excitation microscope module 120 without staining the brain of the subject by invasive methods (such as administration of agent). However, in order to confirm the clear experimental results, in this embodiment, optical images showing the autofluorescence of abnormal tau proteins were generated through the ultrashort pulse laser module 110 and the two-photon excitation microscope module 120 after the mouse brain was stained by an invasive method.

FIG. 4 shows the brain image of the mouse captured using an optical imaging system according to an embodiment of the disclosure.

In FIG. 4, it can be seen that the hippocampus and cerebral cortex of the mouse brain are clearly distinguished in the optical image captured using the optical imaging system 10 according to the embodiment.

FIG. 5 shows an image of Aβ plaques, and an image of p-tau404 in the brain stained with a double-positive anti-human PHF-1 antibody for 3-month-old AD-Tg mice and WT mice.

Referring to FIG. 5, it can be seen that, in the hippocampal region and the cerebral cortex of the AD-Tg (5×FAD) mouse brain, the staining patterns and distributions of Aβ plaques and p-tau404 are clearly observed at each position of the brain. Accordingly, it can be seen that the optical imaging system 10 according to the embodiment can detect amyloid-positive signals even in 3-month-old 5×FAD mice. This is very effective compared to the fact that amyloid-positive signals were not detected in PET images of 3-month-old 5×FAD mice.

In contrast, since no Aβ plaque staining pattern is observed in the brain of the WT mouse, it can be seen that there are no Aβ plaques.

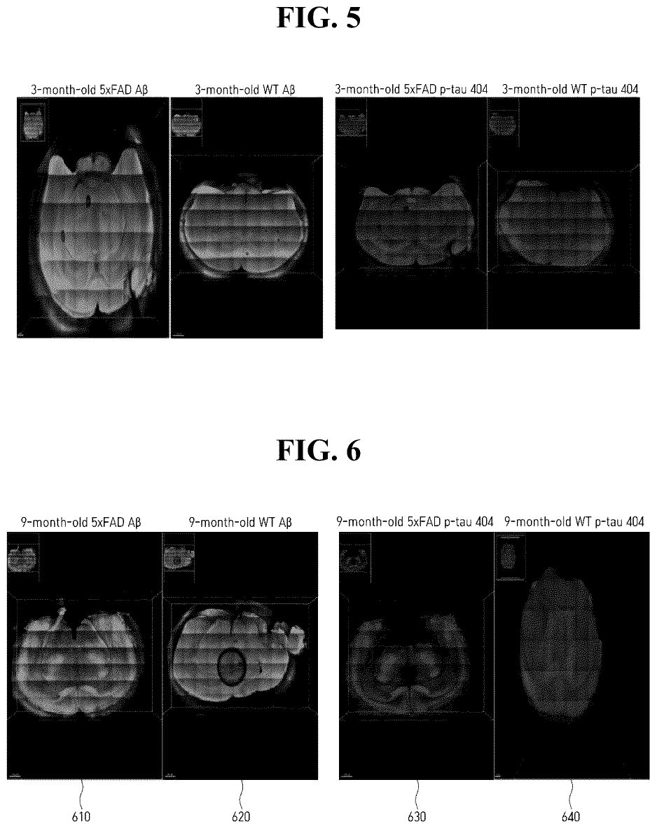

FIG. 6 shows Aβ plaque images 610 and 620 of the brains stained with Thioflavin-S, and p-tau404 images 630 and 640 of the mouse brains stained with a double-positive anti-human PHF-1 antibody for 9-month-old AD-Tg (5×FAD) mice and 9-month-old WT mice.

Referring to FIG. 6, it can be seen that the number of Aβ plaques and their sizes increase in 9-month-old 5×FAD mice compared with 3-month-old 5×FAD mice. In contrast, the WT mice show no Aβ plaque staining pattern, and it can be seen that p-tau404 is not expressed.

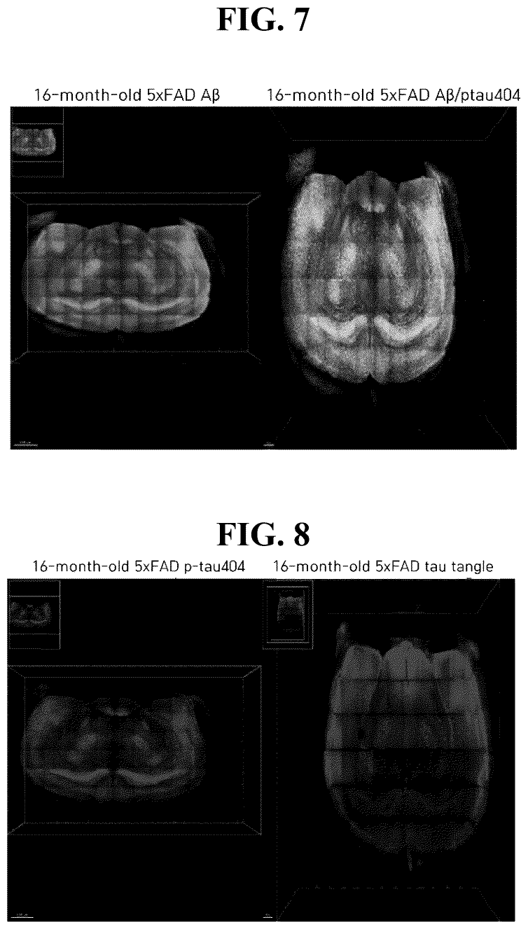

FIG. 7 shows an image obtained by superimposing an Aβ plaque image of the brain of a 16-month-old AD-Tg (5×FAD) mouse stained with Thioflavin-S and a p-tau404 image of the brain stained with a double-positive anti-human PHF-1 antibody.

Referring to FIG. 7, it can be seen that the brain of the 16-month-old 5×FAD mouse has a significantly greater number of Aβ plaques and larger plaque sizes compared with the 9-month-old 5×FAD mouse. When FIGS. 6 and 7 are compared, it can be inferred that the two AD pathological proteins are saturated within the brain.

FIG. 8 shows an Aβ plaque image of the brain stained with Thioflavin-S, and a Tau tangle (NFT) image of the brain stained with a double-positive anti-human Tangle antibody for a 16-month-old AD-Tg (5×FAD) mice.

Referring to FIG. 8, it can be confirmed that, in the optical images of the brains of the 16-month-old 5×FAD mice, Aβ plaques are overexpressed and that tau tangles (NFTs) are also expressed.

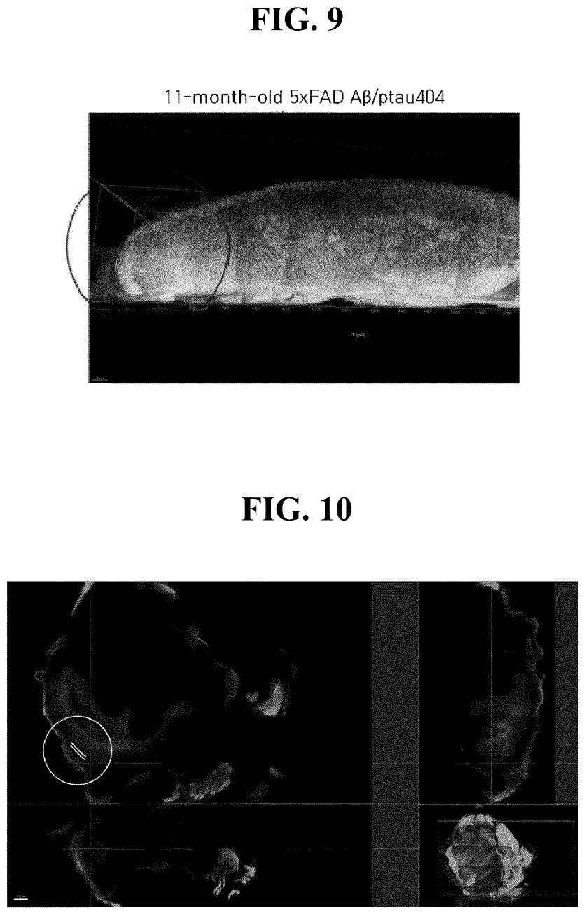

FIG. 9 is a diagram for comparing optical images of Aβ and p-tau404 in the brain of an 11-month-old AD-Tg mouse. In the case of FIG. 9, one optical image for Aβ plaques or p-tau404 overlaps the other optical image for the entire mouse brain as a composite image using the Add Image command of the Imaris software. The one optical image was obtained by capturing the circled portion in FIG. 9 using a commercially available confocal microscope, and the other optical image was obtained by capturing the entire half-brain image of the 11-month-old AD-Tg (5×FAD) mouse using a high-resolution nonlinear optical microscope. However, although the same z-axis thickness was measured, the confocal microscope has a limitation in the depth (z-axis) direction compared with the high-resolution nonlinear optical microscope due to the difference in characteristics between the two microscopes.

Referring to FIG. 9, in the composite optical image, the fluorescent spots in the brain of the AD-Tg mouse appear brighter and more clearly displayed, and thus the presence or absence of Alzheimer's disease can be more clearly distinguished.

FIG. 10 shows an image of a mouse eye captured by the optical imaging system according to an embodiment. In FIG. 10, the image of the mouse eye was captured after the application of a fluorescence staining and tissue clearing process, similar to the mouse brain.

In FIG. 10, the sclera/choroid/retina/vitreous body of the mouse eye are clearly distinguished. The two white lines within the white circle indicate the spacing of the optically transparent retina. The outer layers of the retina form the sclera and the choroid, and the inner side of the retina may be composed of the vitreous body.

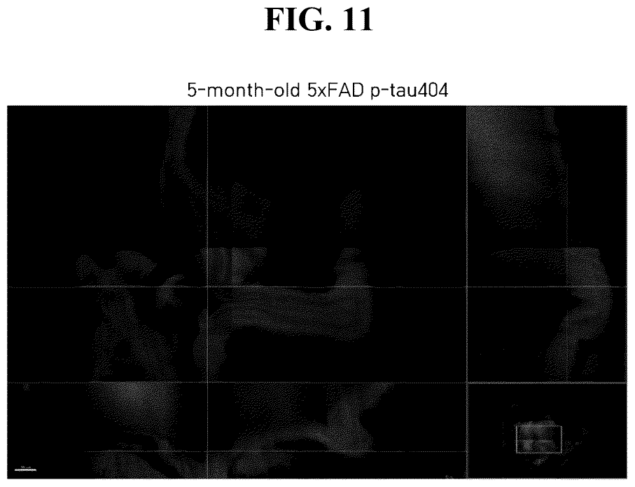

FIG. 11 shows an image of p-tau404 in the retina of a 5-month-old AD-Tg (5×FAD) mouse stained with a double-positive anti-human PHF-1 antibody.

In FIG. 11, an XY section and an XZ/YZ section are displayed on the IMARIS software. In the XY section, it can be seen that a number of p-tau404 fluorescent spots are distributed on the same plane around the position of the cross-hair. In the XZ section and YZ section, it can be seen that the p-tau404 fluorescent spots (see, the bright regions inside the rectangular area in FIG. 11) exist within the retina.

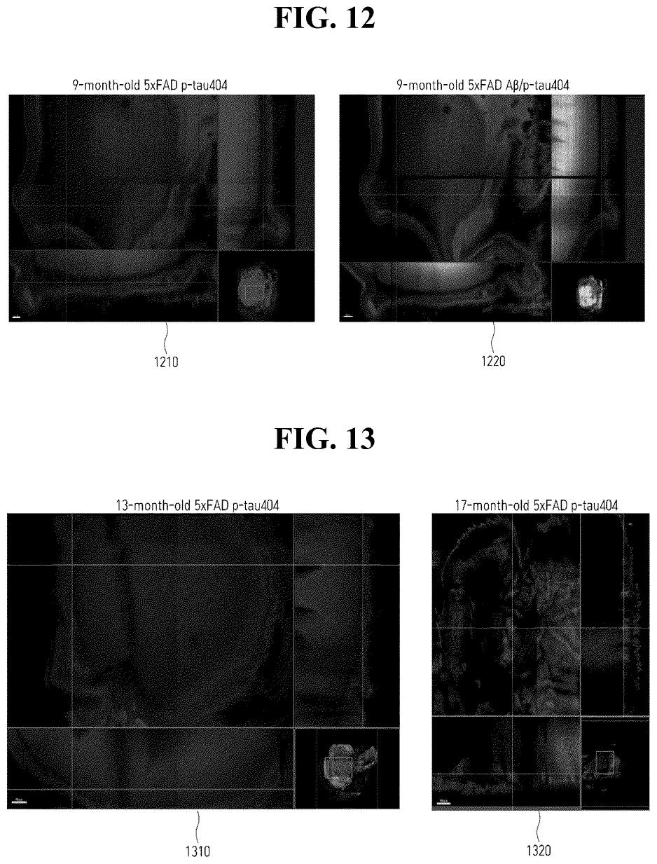

The ocular images of FIGS. 12 and 13 were captured as z-stack images with a thickness of 1.5 mm at intervals of 2 μm, starting from the lowest part of the mouse eye (the position where fluorescence begins to appear).

FIG. 12 shows an image of p-tau404 in the retina of a 9-month-old AD-Tg (5×FAD) mouse stained with a double-positive anti-human PHF-1 antibody. Specifically, a p-tau404 image 1210 in FIG. 12 is a p-tau404 image in the retina of a 9-month-old AD-Tg (5×FAD) mouse stained with a double-positive anti-human PHF-1 antibody, and an Aβ/p-tau404 image 1220 is a composite image in which an Aβ plaque image overlaps the p-tau404 image in the retina stained with a double-positive anti-human PHF-1 antibody.

In FIG. 12, in the composite image 1220, the Aβ plaque fluorescent spots (brighter regions) in the retina and the p-tau404 fluorescent spots stained with the double-positive anti-human PHF-1 antibody are simultaneously displayed.

In this way, similar to the brain optical image of the 9-month-old mouse, it can be seen that Aβ plaque fluorescent spots and p-tau404 fluorescent spots are also observable in the p-tau404 image of the retina.

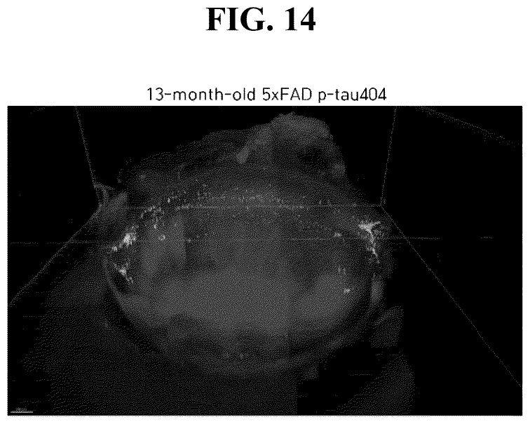

FIG. 13 shows p-tau404 images 1310 and 1320 in the retinae of 13-month-old and 17-month-old AD-Tg mice (5×FAD) stained with a double-positive anti-human PHF-1 antibody. FIG. 14 shows the result of fluorescent spot analysis for a p-tau404 image 1400 in the retina of a 13-month-old AD-Tg (5×FAD) mouse stained with a double-positive anti-human PHF-1 antibody.

Referring to FIG. 13, the number and distribution of the fluorescent spots in the p-tau404 image (blue-marked regions in 1310 and white-marked regions in 1320) can be analyzed using the Imaris 10.2 software (Oxford Instruments).

Referring to FIG. 14, the fluorescent spots (white-marked regions) of the Aβ plaque and the p-tau404 images are distributed throughout the ocular region, and it can be seen that the sizes of the fluorescent spots on the left and right sides are similar, while the sizes in the central portion are relatively smaller than those on the left and right sides.

As described above, the optical imaging system 10 according to the embodiment can provide an optical means capable of identifying Aβ plaques, tau tangles, and microglial cells in vivo, thereby enabling the identification of the number and distribution of Aβ plaques and tau tangles, which are conventionally identified through brain autopsies, in vivo, and a clear diagnosis of Alzheimer's disease.

In addition, since the optical imaging system 10 according to the embodiment can diagnose Alzheimer's disease at an early stage, even from the initial stage (3 months) based on ocular images, an appropriate response according to the progression of Alzheimer's disease can be promptly applied to the patient.

Furthermore, since the optical imaging system 10 according to the embodiment can perform repeated measurements in a non-invasive manner, it can support conducting clinical trials while confirming the effects of Alzheimer's disease therapeutics during the clinical testing process.

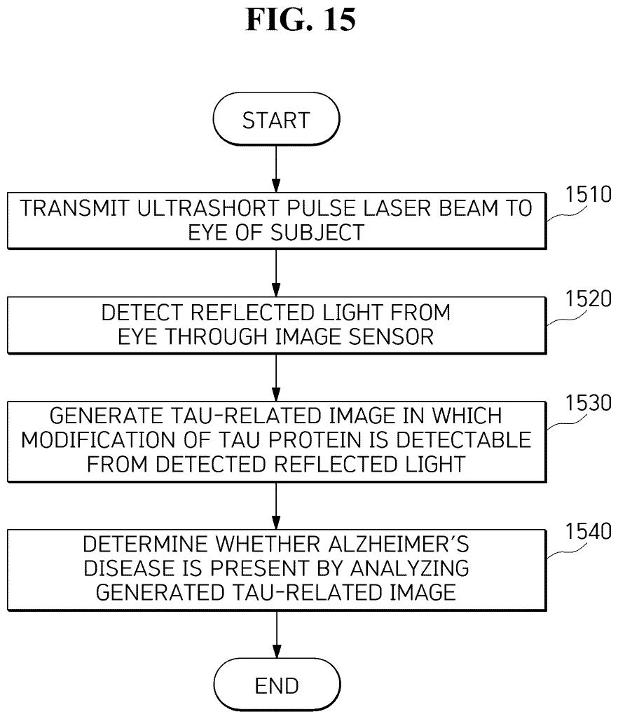

FIG. 15 is a flowchart illustrating an optical imaging analysis method according to an embodiment.

Referring to FIG. 15, in operation 1510, the optical imaging system 10 may radiate an ultrashort pulse laser beam through the laser module 110 and then transmit the radiated laser beam to the eye of a subject through the two-photon excitation microscope module 120.

In operation 1520, the optical imaging system 10 may detect reflected light of the ultrashort pulse laser beam transmitted to the eye or (auto)fluorescence within the eye using the image sensor 125.

In operation 1530, the optical imaging system 10 may generate a tau-related image in which tau protein modification is detectable from the autofluorescence or reflected light detected through the image sensor 125. Here, an examiner may select a wavelength band of the ultrashort pulse laser beam to be used for optical imaging and input a capture timing corresponding to the selected wavelength band. Accordingly, the optical imaging system 10 may selectively filter a laser beam in the selected wavelength band to generate an optical image.

In operation 1540, the optical imaging system 10 may determine whether Alzheimer's disease is present by analyzing the generated tau-related image. Similarly, the optical imaging system 10 may generate an Aβ plaque image or a microglial cell image.

As described above, the optical imaging system 10 according to the embodiment, without staining the Aβ plaques, p-tau, and microglial cell in vivo of the examinee with a biologically invasive method (e.g., by administration of an agent), can generate an optical image that determines whether an examinee has Alzheimer's disease by detecting, through the ultrashort pulse laser module 110 and the two-photon excitation microscope module 120, not only Aβ plaques caused by the ultrashort pulse laser beam but also spontaneous fluorescence of p-tau and microglial cell.

In addition, the optical imaging system 10 according to the embodiment can perform non-invasive examinations and repeated measurements, thereby enabling early diagnosis of Alzheimer's disease, simplifying the overall examination process, and reducing the examination burden on both the subject and the examiner.

Furthermore, the optical imaging system 10 according to the embodiment may be configured at a lower cost than an expensive PET examination device that is implemented in a form for seeing through the skull for brain diagnosis, thereby facilitating commercialization.

According to various embodiments disclosed in the present document, an optical image related to the differentiation of Alzheimer's disease can be generated based on an ultrashort pulse laser beam and two-photon excitation microscope structure. In addition, various effects that can be directly or indirectly identified through the present document may be provided.

It is to be understood that various embodiments of the present document and terms used in the embodiments are not intended to limit technological features set forth herein to specific embodiments and include various modifications, equivalents, or substitutions for the embodiments. With regard to the description of the drawings, similar reference numerals may be used to refer to similar or related components. A singular form of a noun corresponding to an item may include one or more of the items unless the relevant context clearly indicates otherwise. As used herein, each of phrases such as “A or B,” “at least one of A and B,” “at least one of A or B,” “A, B, or C,” “at least one of A, B, and C,” and “at least one of A, B, or C” may include any one of or all possible combinations of items enumerated together in a corresponding one of the phrases. Terms such as “1^{st}” and “2^{nd}” or “first” and “second” may be used to simply distinguish a corresponding component from another, and do not limit the components in other aspects (e.g., importance or order). When a (e.g., first) component is referred to, with or without the term “functionally” or “communicatively,” as “coupled” or “connected” to another (e.g., second) component, it means that the first component may be coupled to the second component directly (e.g., by wire), wirelessly, or via a third component.

As used herein, the term “module” may include a unit implemented in hardware, software, or firmware, and may be interchangeably used with other terms, for example, “logic,” “logic block,” “part,” or “circuitry.” A module may be a single integral component or a minimum unit or part thereof that performs one or more functions. For example, according to an embodiment, a module may be implemented in the form of an ASIC.

Various embodiments of the present document may be implemented as software (e.g., a program) including one or more instructions stored in a storage medium (e.g., an internal memory or an external memory) that is readable by a machine (e.g., the optical imaging system 10). For example, a processor (e.g., processor 137) may invoke at least one of the one or more instructions stored in the storage medium and execute the at least one invoked instruction. This allows the machine to be operated to perform at least one function in accordance with the at least one invoked instruction. The one or more instructions may include code generated by a compiler or code executable by an interpreter. The machine-readable storage medium may be provided in the form of a non-transitory storage medium. Here, the term “non-transitory” simply means that the storage medium is a tangible device, and does not include a signal (e.g., an electromagnetic wave), but this term does not distinguish between a case where data is semi-permanently stored in the storage medium and a case where data is temporarily stored in the storage medium.

According to an exemplary embodiment, a method according to various embodiments disclosed in the present document may be included and provided in a computer program product. The computer program product may be traded as a product between a seller and a buyer. The computer program product may be distributed in the form of a machine-readable storage medium (e.g., a compact disc (CD) read-only memory (ROM)) or distributed (e.g., downloaded or uploaded) online via an application store (e.g., PlayStore™) or directly between two user devices (e.g., smartphones). When the computer program product is distributed online, at least a part of the computer program product may be temporarily generated or at least temporarily stored in the machine-readable storage medium, such as memory of the manufacturer's server, a server of the application store, or a relay server.

Components according to various embodiments of the present document may be implemented in the form of hardware such as a digital signal processor (DSP), an FPGA, or an ASIC and perform certain roles. Components are not limited to software or hardware, and each component may be configured to reside in an addressable storage medium or run on one or more processors. As an example, components may include components such as software components, object-oriented software components, class components, and task components, processes, functions, attributes, procedures, subroutines, segments of program code, drivers, firmware, microcode, circuitry, data, databases, data structures, tables, arrays, and variables.

According to various embodiments, each of the above-described components (e.g., modules or programs) may include a single entity or a plurality of entities. According to various embodiments, one or more of the above-described components or operations may be omitted, or one or more other components or operations may be added. Alternatively, or additionally, a plurality of components (e.g., modules or programs) may be integrated into a single component. In this case, the integrated component may still perform one or more functions of each of the plurality of components in the same or similar manner as they are performed by the corresponding one of the plurality of components before the integration. According to various embodiments, operations performed by a module, a program, or another component may be carried out sequentially, in parallel, repeatedly, or heuristically, at least one of the operations may be executed in a different order or omitted, or one or more other operations may be added.

According to various embodiments disclosed in the present document, it is possible to simplify hardware and control on the basis of a wired RIS control link. In addition, various effects that are directly or indirectly found in the present document can be provided.

What is claimed is:

1. An optical imaging system comprising:
a laser system configured to radiate an ultrashort pulse laser beam;
a microscope module configured to transmit the radiated ultrashort pulse laser beam to an eye of a subject and detect reflected light from the eye or (auto)fluorescence in the eye using an image sensor;
a control module configured to generate a tau-related image in which a modification of tau proteins is detectable from the reflected light detected through the image sensor; and
an output device configured to display the tau-related image.

2. The optical imaging system of claim 1, wherein the laser system radiates the ultrashort pulse laser beam that has multiple wavelengths within a specified wavelength bandwidth, in which a central wavelength ranges from 790 nm to 810 nm.

3. The optical imaging system of claim 2, wherein the specified wavelength bandwidth includes 11 nm.

4. The optical imaging system of claim 2, wherein the laser system radiates laser beams that have a pulse width between 50 fs and 65 fs at a repetition rate between 80 MHz and 110 MHz.

5. The optical imaging system of claim 1, further comprising a wavelength-tunable module,
wherein the control module controls the wavelength-tunable module to selectively filter light of a first wavelength band in which modification of tau protein is detectable from the detected (auto)fluorescence or reflected light,
detects the filtered light of the first wavelength band through the image sensor, and
converts a color of the detected light of the first wavelength band into a color matched to the first wavelength band to generate the tau-related image.

6. The optical imaging system of claim 1, wherein the control module analyzes the number and distribution of p-tau fluorescent spots in the eye in the tau-related image and determines whether the subject has Alzheimer's disease based on an analysis result.

7. The optical imaging system of claim 1, wherein the tau-related image is generated to include a central region and left and right regions of the eye.

8. The optical imaging system of claim 1, wherein the control module further generates an Aβ plaque image in which Aβ plaques are detectable based on the detected reflected light.

9. The optical imaging system of claim 8, wherein the control module selectively filters light of a second wavelength band in which Aβ plaques are detectable from the detected reflected light, detects the filtered light of the second wavelength band through the image sensor, and converts a color of the detected light of the second wavelength band into a second color matched to the second wavelength band to generate the Aβ plaque image.

10. The optical imaging system of claim 8, wherein the control module analyzes the number and distribution of Aβ plaque fluorescent spots in the generated Aβ plaque image and determines whether the subject has Alzheimer's disease based on an analysis result.

11. The optical imaging system of claim 8, wherein the control module generates a composite image in which the Aβ plaque image overlaps the tau-related image and analyzes whether the subject has Alzheimer's disease based on the composite image.

12. The optical imaging system of claim 1, wherein the control module generates a p-tau404 image in which a p-tau404 is detectable when the modification of the tau protein is the p-tau404 and generates a tau tangle image on which tau is fluorescently displayed when the modification of the tau protein is a tau tangle.

13. The optical imaging system of claim 1, wherein the control module selectively filters light of a third wavelength band in which fluorescence of Iba1/AIF-1 protein is detectable from the detected reflected light, detects the filtered light of the third wavelength band through the image sensor, converts a color of the detected light of the third wavelength band into a color matched to the third wavelength band to generate a microglial cell image, and determines whether the subject has Alzheimer's disease based on the microglial cell image.

14. An optical imaging analysis method that is performed by an optical imaging system that includes a laser module, a microscope module, and a control module, the optical imaging analysis method comprising:
radiating an ultrashort pulse laser beam through the laser module and then transmitting the radiated ultrashort pulse laser beam to an eye of a subject through the microscope module;
detecting reflected light from the eye or (auto)fluorescence in the eye generated by the transmitted ultrashort pulse laser beam using an image sensor;
generating a tau-related image in which modification of tau proteins is detectable from the (auto)fluorescence or reflected light detected through the image sensor; and
determining whether Alzheimer's disease is present by analyzing the generated tau-related image.

15. The optical imaging analysis method of claim 14, comprising:
selectively filtering, by a wavelength-tunable filter, light of a first wavelength band in which p-tau404 is detectable from the detected reflected light;
detecting, by the image sensor, the filtered light of the first wavelength band; and
converting a color of the detected light of the first wavelength band into a color matched to the first wavelength band to generate the tau-related image.

16. The optical imaging analysis method of claim 14, wherein the determining of whether Alzheimer's disease is present includes:
analyzing the number and distribution of p-tau fluorescent spots in the eye in the tau-related image; and
determining whether the subject has Alzheimer's disease based on an analysis result.

17. The optical imaging analysis method of claim 14, further comprising generating an Aβ plaque image in which Aβ plaques are detectable based on the detected reflected light.

18. The optical imaging analysis method of claim 17, wherein the generating of the Aβ plaque image includes:
selectively filtering light of a second wavelength band in which the Aβ plaques are detectable from the detected reflected light or (auto)fluorescence;
detecting, by the image sensor, the light of the second wavelength band; and
converting a color of the detected light of the second wavelength band into a second color matched to the second wavelength band to generate the Aβ plaque image.

19. The optical imaging analysis method of claim 17, further comprising generating a composite image in which the Aβ plaque image overlaps the tau-related image,
wherein the determining of whether Alzheimer's disease is present includes analyzing whether the subject has Alzheimer's disease in the subject based on the composite image.

20. The optical imaging analysis method of claim 14, wherein the generating of the tau-related image includes at least one of generating a p-tau404 image in which a p-tau404 is detectable when the modification of the tau protein is the p-tau404 and generating a tau tangle image on which tau is fluorescently displayed when the modification of the tau protein is the tau tangle.
